(19) United States
(12) Patent Application Publication
(10) Pub. No.: US 2023/0053095 A1
(43) Pub. Date: Feb. 16, 2023

(54) METHODS FOR PRODUCING, STORING, AND USING ENERGY

(71) Applicant: King Power Company LLC, Ooltewah, TN (US)

(72) Inventor: Forrest A. King, Ooltewah, TN (US)

(21) Appl. No.: 17/794,111

(22) PCT Filed: Jan. 21, 2021
(86) PCT No.: PCT/US21/14462

§ 371 Date: Jul. 20, 2022

Related U.S. Application Data
(60) Provisional application No. 62/963,901, filed on Jan. 21, 2020.

(51) Int. Cl. C01B 3/48; B01J 8/26; B01J 8/18; C07C 1/12

(52) U.S. Cl. CPC C01B 3/48; B01J 8/26; B01J 8/1827; B01J 8/1836; C07C 1/12; C01B 2203/0233; C01B 2203/0283; C01B 2203/062; C01B 2203/1241; C01B 2203/84; C01B 2203/0861; B01J 2208/00504

(57) ABSTRACT

A series of three chemical reactions, including a combination of endothermic and exothermic reactions, is used to generate, store, and supply on-demand heat from renewable energy sources for use in a variety of processes. Products from one reaction are used in the next reaction, and the series of three reactions is carried out once or more than once, optionally as a closed loop process.

### FIELD

Disclosed herein are methods and processes for producing, storing, and using energy. More particularly, the methods use a series of chemical reactions carried out in sequence to produce and store energy for use on demand.

### BACKGROUND

Most industrial processes require large amounts of heat for various processes. Many industries consume enormous amounts of natural gas which is used as a source of heat. The natural gas generally is burned, sending large amounts of CO₂ into the atmosphere. A need exists to produce large amounts of heat for use in industrial and other environments in an environmentally friendly and cost-effective manner.

### SUMMARY

Described herein are methods for generating, storing, and using heat energy on demand. The methods include performing a series of chemical reactions, comprising at least one endothermic reaction and at least one exothermic reaction, wherein each reaction involves reacting a product from the preceding reaction in the series (if any), and wherein optionally at least the first reaction is repeated after each reaction in the series has been carried out. The method can be used to store renewable energy for use on-demand, as will be described in detail herein.

### BRIEF DESCRIPTION OF THE FIGURES

FIG. 1 is a schematic of the basic equipment for carrying out the chemical reactions of the methods disclosed herein.

FIG. 2 is a schematic of the basic equipment for carrying out the chemical reactions of the methods disclosed herein.

FIG. 3 is a schematic of a system (500) for carrying out the methods described herein.

### DETAILED DESCRIPTION

Described herein are methods for generating, storing, and using heat energy on demand. The methods produce and use methane, hydrogen, and other reactants. The methods use a combination of endothermic and exothermic reactions to generate potential heat energy for immediate use or for storage and later use on demand. The heat energy also is suitable for a variety of uses such as generating electricity. The methods do not require combustion or carbon dioxide emissions.

Methods are provided for generating and optionally storing potential heat energy and producing useable heat energy on demand. The methods include performing a series of chemical reactions, comprising at least one endothermic reaction and at least one exothermic reaction, wherein each reaction involves reacting a product from the preceding reaction in the series (if any), and wherein at least the first reaction is repeated after each reaction in the series has been carried out. In some examples, a product of at least one endothermic reaction optionally is stored for a period of time, thereby storing potential heat energy, before that product is reacted in at least one exothermic reaction, thereby producing useable heat energy. In some examples, the entire series of chemical reactions is carried out more than once, more than twice, or more than three times. The series of reactions can be carried out as a closed loop, as a partially closed loop, or as an open loop, as explained further herein.

The methods includes performing the following reactions in series:

CH₄+H₂O→CO+3H₂ I

CO+H₂O→CO₂+H₂ II

CO₂+4H₂→CH₄+2H₂O III

Reaction I has an enthalpy of reaction, ΔH°_{r×n}, of 206 kJ/mol, so it is endothermic. Reaction II also has an enthalpy of reaction, ΔH°_{r×n}, of −41 kJ/mol, so it is exothermic. And Reaction III has an enthalpy of reaction, ΔH°_{r×n}, of −165 kJ/mol, so it is exothermic. Reactions I, II, and III are sometimes referred to in the art by the names “steam methane reformation,” “water-gas-shift,” and “Sabatier reaction,” respectively. For convenience, Reactions I, II, and III are sometimes referred to herein by those names or the abbreviations “SMR” (Reaction I), “WGS” (Reaction II), and “SAB” (Reaction III). Critically, however, those names and abbreviations are used as a matter of convenience only to identify the reactants and products involved, and the use of those names and abbreviations does not indicate use of any particular reaction conditions (e.g., catalyst, temperature, pressure) or equipment (e.g. reactor). The reaction conditions and equipment for Reactions I, II, and III are expressly disclosed herein.

Any reaction in the series may be performed first in the series, provided the three reactions are performed in one of the following orders: I, II, III; II, III, I; III, I, II and provided at least a portion of a product from each reaction is used as a reactant in the following reaction. Optionally, after the series of reactions is carried out once, the first step is carried out a second time. Optionally after the first step is carried out a second time, the second step is carried out a second time. Optionally, the entire series of reactions may be repeated one or more times. Optionally, a product of any of Reactions I, II, or III can be stored for a period of time before the subsequent reaction is carried out. Storing a product of Reaction I or Reaction II stores potential heat energy in the chemical bonds of the product compounds. Potential heat energy stored in the products of Reaction II is converted to useable heat in exothermic Reaction III.

Thus, in some examples, a method for generating and storing energy for use on demand, includes (i) carrying out a series of reactions consisting of Reactions I, II, and III

CH₄+H₂O→CO+3H₂ (I)

CO+H₂O→CO₂+H₂ (II)

CO₂+4H₂→CH₄+2H₂O (III)

wherein any of Reaction I, II, or III can be a first reaction in the series, provided when Reaction I is the first reaction in the series, at least some of the CO produced in Reaction I is reacted in Reaction II, and at least some of the CO₂ produced in Reaction II is reacted in Reaction III; when Reaction II is the first reaction in the series, at least some of the CO₂ produced in Reaction II is reacted in Reaction III, and at least some of the CH₄ produced in Reaction III is reacted in Reaction I; and when Reaction III is the first reaction in the series, at least some of the CH₄ produced in Reaction III is reacted in Reaction I, and at least some of the CO produced in Reaction I is reacted in Reaction II; and (ii) repeating the first reaction in the series of reactions.

In some examples, when Reaction I is the first reaction in the series of reactions, step (ii) comprises reacting at least some of the CH₄ produced in Reaction III in Reaction I. In other examples, when Reaction II is the first reaction in the series of reactions, step (ii) comprises reacting at least some of the CO produced in Reaction I in Reaction II. In still other examples, when Reaction III is the first reaction in the series of reactions, step (ii) comprises reacting at least some of the CO₂ produced in Reaction II in Reaction III.

Optionally, the method further comprises after repeating the first reaction, repeating the second reaction. Optionally, the method further comprises after repeating the second reaction, repeating the third reaction. Optionally, the method further comprises after repeating the third reaction, repeating the series of reactions one or more times.

An important advantage of the invention is that a renewable energy source can be used for the energy put into the system. When a renewable energy source is used to drive the endothermic reactions in the series, the exothermic reactions produce heat that is derived from a renewable source, even if no renewable energy source is available at the location or time when the reaction is carried out and the heat is produced. Thus, the methods described herein make it possible to take advantage of available renewable energy even at a time or place removed from the source of the renewable energy. That is, the methods can be used to generate and store potential heat energy for use at a later time and/or at a different location. Moreover, the heat energy produced by the exothermic reactions optionally can be converted to electrical energy. In that way, the methods described herein can provide heat and/or electrical energy from a renewable energy source at a time and/or location where a source of renewable energy is not available, is not convenient to access, or does not produce an adequate of energy.

Another advantage of using one or more renewable energy sources in the method is that in some examples, the entire method can be carried out without carbon dioxide emissions.

Systems and methods disclosed herein make use of one or more advantageous underlying design principles. For example, the systems and methods use fluidized bed reactors (“FBR”s), which allow rapid mixing and provide excellent heat and mass transfer between the reactant gases and the solid catalysts that facilitate the reactions. As another example, the systems and methods use insulated equipment (primarily vacuum insulated), including but not limited to, insulated reactors, pipes, lines, vessels, and tanks, which aid in heat management and transfer. As a further example, the systems and methods can be carried out as a partially or entirely closed loop processes, which aids in stabilizing reactants (e.g., CO). Together, these underlying design principles provide safe and rapid reactions with catalytic conversion to desired outcomes on a reproducible basis.

The methods can be carried out as a closed loop process. The term “closed loop process” as used herein means that no reactant or product, i.e., CO, Hz, CO₂, H₂O, or CH₄, is added or removed during the method. So, after the system is charged with initial reactants, each reaction yields products that are used as reactants in the next reaction in the series, and the series of reactions is carried out one or more times without introducing additional reactant or product and without removing any product or reactant. In some examples, the method can include carrying out multiple iterations of the series of reactions (i.e. repeating the series one or more times) without adding or removing reactant or product.

Energy may be added to or removed from the system, e.g., in the form of heat or electricity, even if the system is a closed loop system. For example, energy may be added to initiate a reaction or to drive a reaction forward. Similarly, energy may be removed, for example, for use in another system or process. For example, heat produced by exothermic Reaction III can be captured and used for any desired purpose. Additionally or alternatively, if heat is supplied to any part of the system in excess of what is needed, that excess heat also can be captured and used for any desired purpose. Energy also can be added or removed as necessary to store products for a period of time and/or to transport products to a different location before a subsequent reaction (as described further herein) or for any other purpose.

Also, it should be noted that product and reactant gases can be routed through additional equipment such as heat exchangers and electricity generating turbines even as part of a closed system, provided no product or reactant is added or removed. Thus, the product and reactant gases can be directed along a path, stored, or transported without removing reactant or product or interrupting the closed loop system.

At any point in the process, the product gases can be stored for a period of time before being used as reactants in the next reaction. Thus, the method can be used to store renewable energy for use on-demand, as will be described in detail herein.

In some examples, the product of one or more reactions advantageously can be stored before a subsequent reaction for a period of time of at least 6 hours, at least 12 hours, at least 24 hours, or at least 48 hours. When the products are stored before the next reaction, they can be retained in any convenient manner. For example, the system can include at least one storage vessel between adjacent reactors. The storage vessel(s) can be in fluid communication with the reactors, so the products/reactants can be stored without opening a closed system. The storage vessel can be selected to maintain the stored products/reactants at any temperature, such as the reaction temperature at which they were produced, another elevated temperature, ambient temperature, or a reduced temperature. The storage vessel can be selected to maintain the stored products/reactants at any pressure, such as ambient or reduced pressure.

In some examples, the method further comprises storing for a period of time at least a portion of the CO, H₂, CO₂, H₂O, or CH₄ after it is produced by Reaction I, II, or III and before it is reacted in the next reaction. In some examples, the CO and/or H₂ is stored for a period of time that exceeds 6 hours, 12 hours, or more than 24 hours. The products can be stored in a tank or another vessel, optionally under compression and/or optionally under reduced temperature.

In some examples, at least a portion of the CO or the H₂ is stored after it is produced by Reaction I and before it is reacted in Reaction II. In other examples, at least a portion of the CO₂ is stored after it is produced by Reaction II and before it is reacted in Reaction III. In still other examples at least a portion of the CH₄ is stored after it is produced by Reaction III and before it is reacted in Reaction I. Any product can be stored for a period of time that exceeds 6 hours, 12 hours, or more than 24.

Still another advantage of the methods described herein is that at any point in the process, the product gases can be transported to another location being used as reactants in the next reaction. For example, the method optionally further comprises transporting at least a portion of the CO, Hz, CO₂, H₂O, or CH₄ after it is produced by Reaction I, II, or III and before it is reacted in the next reaction, wherein the transporting is over a distance of less than 5 miles, less than 1 mile, less than 500 feet, less than 100 feet. Alternatively, the method further comprises transporting at least a portion of the CO, Hz, CO₂, H₂O, or CH₄ after it is produced by Reaction I, II, or III and before it is reacted in the next reaction, wherein the transporting is over a distance of more than 5 miles, more than 10 miles, more than 100 miles, or more than 500 miles. Any product can be transported by any convenient means, including but not limited to pipeline, rail, tube trailer, or any other suitable transportation method, or combination of methods

In some examples, at least a portion of the CO or the H₂ is transported after it is produced by Reaction I and before it is reacted in Reaction II. In other examples, at least a portion of the CO₂ is transported after it is produced by Reaction II and before it is reacted in Reaction III. In still other examples at least a portion of the CH₄ is transported after it is produced by Reaction III and before it is reacted in Reaction I. Any product can be transported by any convenient means, including but not limited to pipeline, rail, tube trailer, or any other suitable transportation method, or combination of methods.

One important advantage of the methods described herein is the ability of the methods to provide energy derived from a renewable energy source at a time and/or location where a renewable energy source would not otherwise be available.

Advantageously, a renewable energy source is used to provide heat that is added to the endothermic reactions to initiate the reactions and/or drive the reactions forward. That renewable energy can be “stored” by retaining the carbon dioxide and hydrogen produced by Reaction II for a period of time before reacting them in Reaction III. Storing the carbon dioxide and hydrogen effectively stores the heat put into the product gases. Advantageously, the carbon dioxide and hydrogen can be stored for a period of time of at least 6 hours, at least 12 hours, at least 24 hours, at least 48 hours, or whenever the stored energy is needed. The stored renewable energy can then be accessed on-demand by reacting the carbon dioxide and hydrogen in exothermic Reaction III. The heat produced Reaction III can be used directly, as heat, or can be converted to another form of energy such as electricity.

Additionally or alternatively, the carbon dioxide and/or hydrogen produced by Reaction II can be transported to a location where no renewable energy source exists or where any renewable energy source is insufficient, inaccessible, or impractical. Transporting the carbon dioxide and hydrogen effectively transports the renewable energy from one location to another. In some examples, Reaction II can be carried out at a location where renewable energy is abundant and the carbon and/or the hydrogen produced by that reaction can be transferred to an area where renewable energy is not as available. Advantageously, the carbon dioxide and/or hydrogen can be transported to a location several miles or several thousands of miles away, or wherever the stored energy is needed. The transported renewable energy can then be accessed on-demand by reacting the carbon dioxide and hydrogen in exothermic Reaction III. The heat produced Reaction III can be used directly, as heat, or can be converted to another form of energy such as electricity.

After accessing the renewable energy through Reaction III, the methane and steam produced by Reaction III can be used to repeat the process of storing and using heat. Another advantage of the methods is that the methane and steam produced by Reaction III also can be stored for a period of time and/or transported to a different location before they are reacted in Reaction I. For example, if Reaction III is carried out to produce heat because no source of renewable energy is available, after the methane and steam are produced by Reaction III, the methane and/or steam can be stored until a renewable energy source is available to initiate and/or drive endothermic Reaction I. Additionally or alternatively, the methane and/or steam can be transported to a location where a renewable energy source is available to initiate and/or drive endothermic Reaction I. Thus, the process of storing and accessing energy can be repeated immediately, or after a period of time, such as at least 6 hours, at least 12 hours, at least 24 hours, at least 48 hours, or whenever a renewable energy source is available. And the process of storing and accessing energy can be carried out at a single location or at multiple locations, depending on where the renewable energy source is located and where the energy is needed.

The energy provided to the system can be derived from wind, solar, hydro, geothermal, tidal energy, or any other renewable energy source. One example of a heat source that is useful for initiating and driving the necessary chemical reactions is plasma, and the plasma can be derived from a renewable energy source. In some examples of the methods described herein, plasma is used as a heat source to provide the heat required to initiate or maintain a chemical reaction and the plasma is derived from a renewable energy source. For example, plasma heat can be used in an outer vessel surrounding one of the fluidized bed reactors to imitate or to drive the reaction. The plasma system envisioned will generate plasma from water, it will not be explosive and not require a carrier gas. Moreover, a plasma heater can be powered by renewable energy/electricity, as well as by a turbine powered by excess heat energy from the reaction system. The waste heat of the plasma vessel could also be used to run a turbine as previously described. elsewhere in the reaction system, which would increase efficiency of the overall system. The temperatures accessible using plasma exceed all heat requirements for the methods described herein, so necessary input temperatures for any point in the process can be readily reached. Thus, plasma derived from a renewable energy source can be used to provide the heat to initiate or maintain one or more of the chemical reactions. Moreover, since the temperatures of the plasma exceed the requirements of the present methods, the waste heat from the plasma can be utilized at multiple points in the process and/or can be recycled to greatly enhance the overall efficiency of the methods, for example, by reducing parasitic heat losses and other heat losses which will occur along the various steps of the reaction pathways, and/or to produce electricity as previously described.

Using a renewable energy source to drive the endothermic chemical reactions, converts the renewable energy to bond energy, which allows capture and storage of that energy for later use.

The methods described herein can be used to generate energy for any desired purpose. For example, thermal energy can be captured and used to heat another system, device, or fluid, or the thermal energy can be converted to electricity.

Each of the three reactions involved in the method takes place at elevated temperature. Thus, thermal energy can be captured from any hot product gas mixture. Reaction III, and the product gas mixture produce by Reaction III, however, will provide the most thermal energy, due to the highly exothermic nature of the reaction. In some examples, a hot product gas mixture has an initial temperature from about 400° C. to about 520° C. In other examples, the gas mixture has a initial temperature from about 405° C. to about 515° C., from about 410° C. to about 510° C., or from about 420° C. to about 500° C.

The product gas mixtures also are under elevated pressure at the conclusion of the reactions. In some examples, a product gas mixture has pressure from about 500 psi to about 1000 psi, from about 550 psi to about 900 psi, from about 600 psi to about 850 psi, or from about 650 psi to about 800 psi.

In some examples, the methods described herein can include introducing at least a portion of the hot product gas mixture from Reaction III (or another hot gas mixture) to a conduit or vessel where heat from the hot gas mixture can be transferred to another gas, a liquid, or a solid. For example, at least a portion of the hot gas mixture can be introduced to a steam accumulator to maintain or increase the temperature of steam inside the accumulator. The reaction gas mixture, however, does not mix with the steam in the accumulator. The hot product gas mixture alternatively may be introduced to an accumulator that includes one or more gases in addition to, or instead of, steam. The accumulator can be any vessel that can store gas/heat, and may be fed or filled by one or more streams of gas.

In some examples, in addition to heating one or more gases in an accumulator, a process described herein further includes introducing a portion of the accumulated and heated gas to a turbine generator to convert the thermal energy to electricity. Optionally, the hot product gas mixture from Reaction III (or another hot gas mixture) can be introduced to a turbine generator. Various additional steps optionally can be employed to provide efficient generation of energy by the turbine generator. In some examples, the hot product gas mixture is only one gas introduced to the turbine generator, and the process further includes introducing an additional gas to the turbine generator. In some cases, the additional gas includes steam.

In some examples, after thermal energy is captured from the hot product gas, the product gas is routed to the next reaction in the series. In other examples, after thermal energy is captured from the hot product gas, the product gas is stored in a containment vessel for a period of time before the next reaction in the series. In some examples, a first portion of the hot product gas mixture is routed to or through the accumulator and then to the steam generator and a second portion of the hot gas mixture is routed to or through the accumulator and then to the next fluidized bed reactor.

In some examples of the methods described herein, multiple iterations of the series of reactions are carried out as a partially closed loop system. In a partially closed loop system, the series of reactions is carried out at least once as a closed loop series, i.e., without adding or removing reactant or product. Following at least one closed loop series, another iteration of the series is carried out in which at least one reactant or product is added or removed. Optionally, an iteration of the series that includes a reactant or product being added or removed can be followed by another iteration of a closed loop series. Another example of a partially closed loop process includes carrying out the series of reaction with only water/steam added or removed during the process.

As one example, optionally at least a portion of the carbon monoxide and hydrogen gas mixture produced by Reaction I can be introduced to a Fischer-Tropsch process to synthesize liquid hydrocarbon fuels. This could be done after one or more iterations of the series of reactions, where any of the previous iterations optionally can be carried out as a closed loop system.

As another example, optionally at least some of the hydrogen produced by Reaction I or II can be separated from the other product gas and sold or used in other processes. As still another example, optionally carbon dioxide produced by Reaction II can be separated from the other product gas and sold or used in other processes. Separation and use or sale of the hydrogen and/or carbon dioxide could be done after one or more iterations of the series of reactions, where any of the previous iterations optionally can be carried out as a closed loop system.

Also disclosed herein are systems for use in practicing the methods disclosed herein. The systems are described with reference to the relevant Figures.

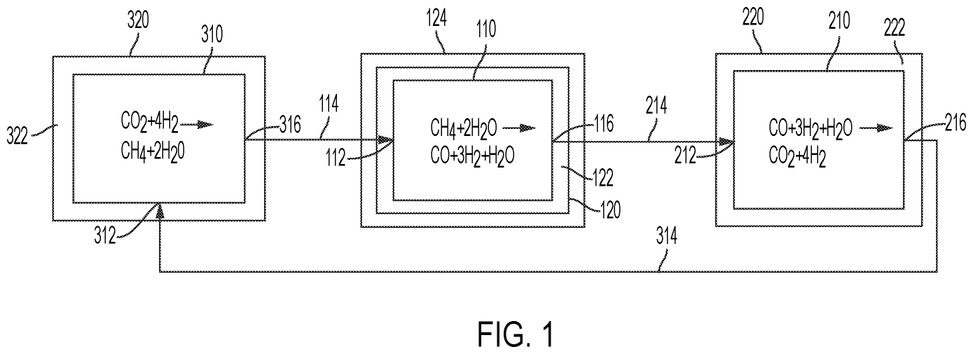

FIG. 1 is a FIG. 1 is a schematic of the basic equipment for carrying out the chemical reactions of the methods disclosed herein. A SMR FBR (110) includes an inlet (112) for receiving SMR reactants (114), an outlet (116) for venting SMR product gases (214), and a catalyst (not shown). The SMR FBR (110) is located within a SMR pressure vessel (120), the interior of which defines a SMR external reactor environment (122). The interior volume of the SMR FBR (110) is not in fluid communication with the SMR external reactor environment (122). The SMR pressure vessel (120) is operable to withstand both elevated and reduced pressures (e.g., vacuum). In some examples, the pressure vessel comprises a vacuum jacket. (124)

Any FBR currently known in the art may be used as the SMR FBR (110). The SMR catalyst can be any catalyst operable to catalyze the reaction of methane with water to form carbon monoxide and hydrogen at the temperatures and pressures provided. In some examples, the SMR catalyst can comprise platinum, palladium. In some examples, the catalyst comprises a commercially available steam-methane reforming catalyst.

A WGS FBR (210) includes an inlet (212) for receiving WGS reactants (214) (which are be the same as the SMR product gases (214)), an outlet (216) for venting WGS product gases (314), and a catalyst (not shown). The WGS FBR (210) is located within a WGS pressure vessel (220), the interior of which defines a WGS external reactor environment (222) the interior volume of the WGS FBR (210) is not in fluid communication with the WGS external reactor environment (222). The WGS pressure vessel (220) is operable to withstand both elevated and reduced pressures (e.g., vacuum). In some examples, the pressure vessel comprises a vacuum jacket (not shown).

Any FBR currently known in the art may be used as the WGS FBR (210). The WGS catalyst can be any catalyst operable to catalyze the reaction of methane with water to form carbon monoxide and hydrogen at the temperatures and pressures provided. In some examples, the WGS catalyst can comprise platinum, palladium. In some examples, the catalyst comprises a commercially available water-gas-shift catalyst.

A SAB FBR (310) includes an inlet (312) for receiving SAB reactants (314) (which can be the same as the WGS product gases (314), an outlet (316) for venting SAB product gases (114) (which can be the same as the SMR reactants (114)), and a catalyst (not shown). The SAB FBR (310) is located within a SAB pressure vessel (320), the interior of which defines a SAB external reactor environment (322). The interior volume of the SAB FBR (310) can be alternately closed off from and opened to the SAB external reactor environment (322), such as by a valve (not shown). The SAB pressure vessel (320) includes an outlet (that can be opened and closed) for venting the SAB product gases (114). Optionally, the SAB FBR (310) can be configured so the reactants can make one or more passes through the SAB FBR (310) before being vented into the SAB external reactor environment (322). The SAB pressure vessel (320) is operable to withstand both elevated and reduced pressures (e.g., vacuum). In some examples, the pressure vessel comprises a vacuum jacket (not shown).

Any FBR currently known in the art may be used as the SAB FBR (310). The SAB catalyst can be any catalyst operable to catalyze the reaction of carbon dioxide with hydrogen to form methane at the temperatures and pressures provided herein. In some examples, the SAB catalyst can comprise platinum or palladium.

Persons skilled in the art will understand that the system shown in FIG. 1 also can include auxiliary equipment such as but not necessarily limited to pumps, valves, gauges, compressors, heat exchangers, and containment vessels. Such auxiliary equipment is not shown in the figures, but persons skilled in the art would understand how and where to incorporate such auxiliary equipment to carry out the methods disclosed herein. In particular, any conduit may be equipped with a check valve or other device to prevent backflow of the gas mixtures. Moreover, all or some of the equipment is insulated to retain heat provided to and produced by the reactions, except where that heat is intentionally diverted for use in a separate application.

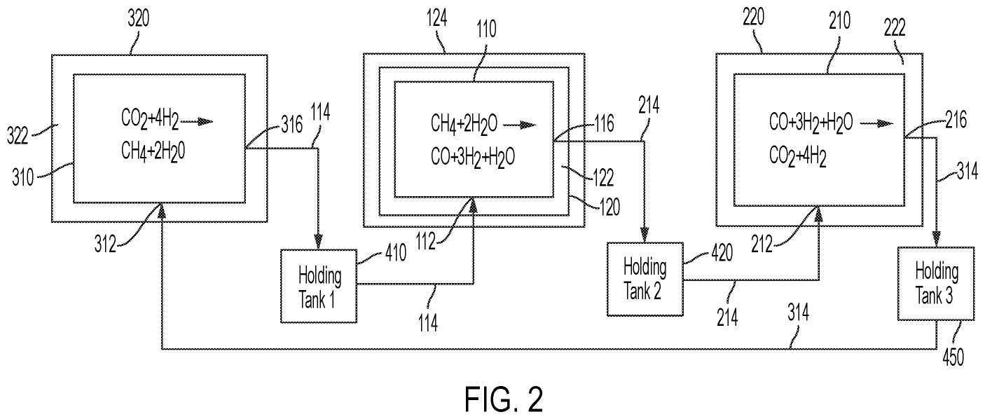

FIG. 2 is a schematic of a system similar to that in FIG. 1 that further includes containment vessels (410, 420, 450) between each reactor for optionally storing the product gas mixture of a reaction before reacting the gas mixture (or a portion thereof) in the next reaction. The containment vessels allow the three reactions in the series to be carried out at two or more different times, as described elsewhere herein.

Persons skilled in the art will understand that energy, e.g. heat, will need to be added to the systems shown in FIGS. 1 and 2 to initiate a reaction and/or to drive the endothermic reactions to completion. Each of the reactions is carried out at elevated temperature. When product gases are stored for subsequent reaction at a later time, depending on the timeframe and storage conditions, those gases may retain some of their heat. Also, however, it may be necessary to supply additional heat to “restart” the series of reactions after a period of delay. Accordingly, one or more heat sources is used in the system to initiate or drive the reactions. Moreover, when the first reaction is initiated, or after a delay in the process (where a gas is stored before subsequent reaction), the pressure in a given piece of equipment may change from an initial pressure to a final pressure, or a steady state pressure.

The systems shown in FIGS. 1-2 can further include certain equipment for providing energy to the system and utilizing energy from the system. For example, after Reaction II two electricity generating turbines could be added, one for the outer shell vessel, and one for the products of the reaction. Step III could also have an added electricity generating turbine. Because of the scale of the system envisioned due to the usage of fluidized bed reactors, and hot gases under pressure, this would be feasible and improve efficiency. Additionally, the turbines would serve to not only generate valuable electricity, but also would tacitly act as “heat exchangers” to balance the heat between the reaction sequences as described

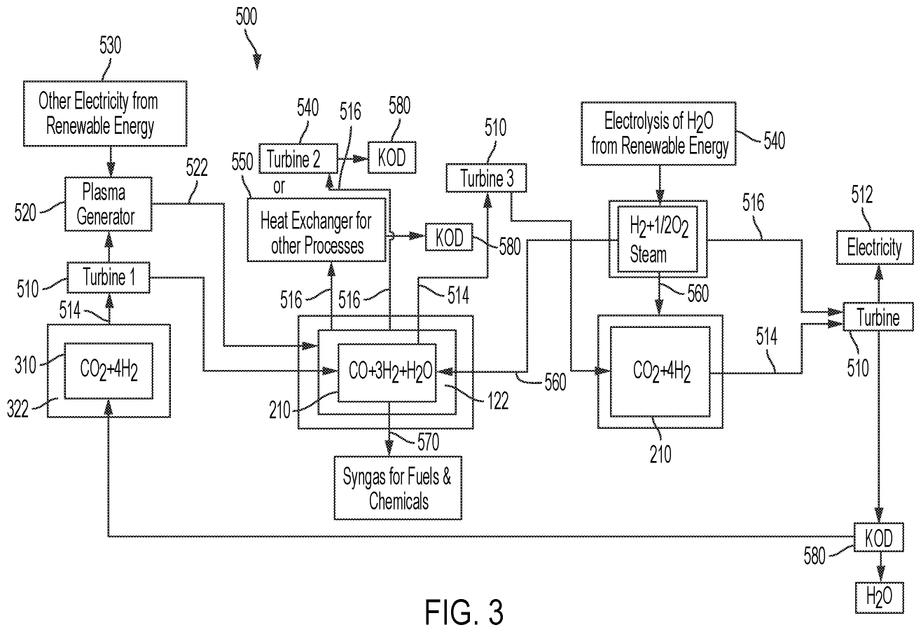

FIG. 3 is a schematic of a system (500) for carrying out the methods described herein. In addition to the equipment identified in FIGS. 1 and 2, which (if shown in FIG. 3) have the same numbers in FIG. 3, FIG. 3 includes a variety of energy sources and energy conversion devices. The system (500) includes turbines (510) for energy conversion, including producing electricity (512) from thermal energy in hot product gases (514) and/or other hot process gases (516); a plasma generator (520) for generating heat (522) from a renewable energy source (530), an electrolyzer (540) powered by renewable energy (not shown); and a heat exchanger (550) for receiving hot product gases (516). FIG. 3 also includes steam lines (560) into WGS FBR (210) and SMR external reactor environment (122), and a line out (570) to collect syngas from WGS FBR (210). FIG. 3 further includes knock out drums (580) for recovering water from the system.

FIG. 3 depicts how the various gases move through move through three appropriately catalyzed vacuum jacketed fluidized bed reactors. The output of the reactors and the output of the plasma generator all go directly through turbines to produce electricity or heat in a closed loop with no CO₂ emissions. The heat and/or electricity can be used in many applications and are not limited to just the applications discussed. After R×n (I) the closed loop optionally can be “opened” as desired so that syngas and hydrogen can proceed to Fischer-Tropsch type reactors to produce a variety of fuels and chemicals. In the example shown, the system includes 3 fluidized bed reactors with associated piping and controls, 4 electricity generating turbines, 1 plasma generator where the plasma is derived from water (and associated plasma nozzles) to provide heat, 3 knockout drums, 1 hydrogen tank/steam (high heat and pressure), and a tank/reactor to start Fischer-Tropsch process for fuels, chemicals, and hydrogen. Those skilled in the art will understand when an alternate piece of equipment is a suitable substitute for the named equipment. Potable water can be obtained if desired.

The following describes non-limiting examples for carrying out the methods disclosed herein. The following examples make reference to the numbered elements shown in the Figures, but are not intended to be limited by any particular Figure. Likewise, the following examples should not be considered to limit the embodiments shown in the Figures.

A SMR reaction includes introducing the SMR reactants (114) to an internal volume of a SMR FBR (110), flowing the SMR reactants (114) through the internal volume of the SMR FBR (110) and over a SMR catalyst (not shown) within the SMR FBR (110) to cause a reaction between the methane and water vapor to form a WGS reactants (214) comprising carbon monoxide and hydrogen, and removing the WGS reactants (214) from the SMR FBR (110). In some examples, the WGS reactants (214) comprises carbon monoxide and hydrogen in a 1:3 molar ratio.

The SMR FBR (110) is located within a SMR external reactor environment (122) that can be defined by an interior of a SMR pressure vessel (120), wherein the interior volume of the SMR FBR (110) is not in fluid communication with the SMR external reactor environment (122). The SMR pressure vessel (120) is operable to withstand both elevated and reduced pressures (i.e., vacuum). In some examples, the pressure vessel comprises a vacuum jacket. In some examples, before the SMR reactants (114) is introduced to the SMR FBR (110), the SMR external reactor environment (122) is heated to a reaction temperature of about 800° C. to about 850° C.

Any FBR currently known in the art may be used as the SMR FBR (110). The SMR catalyst can be any catalyst operable to catalyze the reaction of methane with water to form carbon monoxide and hydrogen at the temperatures and pressures provided. In some examples, the SMR catalyst can comprise platinum, palladium. In some examples, the catalyst comprises a commercially available steam-methane reforming (SMR) catalyst.

A WGS reaction includes introducing at least a portion of the WGS reactants (214) to an internal volume of a WGS FBR (210), and flowing the WGS reactants (214) through the internal volume of the WGS FBR (210) over a WGS catalyst (not shown) to cause a selective catalyzed reaction between carbon monoxide and water to produce a SAB/water vapor gas mixture comprising carbon dioxide, hydrogen, and any remaining water. Any FBR currently known in the art may be used. The WGS catalyst can be any catalyst operable to catalyze the reaction of carbon monoxide with hydrogen and water to form carbon dioxide, hydrogen, and water at the temperatures and pressures provided. In some examples, the WGS catalyst can comprise a commercially available catalyst. The reaction is exothermic.

The WGS FBR (210) is located within a WGS external reactor environment (222) defined by an interior of a WGS pressure vessel (220). The interior volume of the WGS FBR (210) is not in fluid communication with the WGS external reactor environment (222).

In an alternate embodiment, the SMR FBR (110) and the WGS FBR (210) may both be positioned within the interior space of the SMR pressure vessel (120).

A SAB reaction includes introducing SAB reactants (314) comprising carbon dioxide and hydrogen, individually or as a mixture, to an internal volume of a SAB FBR (310); flowing the carbon dioxide and hydrogen over a SAB catalyst (not shown) within the SAB FBR (310) to cause a reaction between the carbon dioxide and the hydrogen, where the reaction produces SMR reactants (114) comprising methane and water vapor.

Any FBR currently known in the art may be used as the SAB FBR (310). In some examples, the internal volume of the SAB FBR (310) can be alternately closed off from and opened to the environment surrounding the reactor, such as by a valve (not shown). Optionally, the SAB FBR (310) can be configured so the reactants can make one or more passes through the SAB FBR (310).

The SAB catalyst can be any catalyst operable to catalyze the reaction of carbon dioxide with hydrogen to form methane at the temperatures and pressures provided herein. In some examples, the SAB catalyst can comprise platinum or palladium.

Optionally, the SAB FBR (310) is housed within an enclosed space within an enclosed vessel, such as a pressure vessel. Optionally, the SMR reactants (114) is vented from the internal volume of the SAB FBR (310) into the enclosed space within the SAB pressure vessel (320). Optionally, the SAB pressure vessel (320) can remain closed with the SMR gas accumulating inside the vessel until the pressure of the SMR reactants (114) in the enclosed space reaches a desired final pressure. The residence time within the SAB FBR (310) can be controlled by closing off the internal volume of the SAB FBR (310) from the enclosed space within the SAB pressure vessel (320) so that gas cannot vent into the vessel, but recirculates through the fluidized bed before being vented into the vessel.

When the SAB FBR (310) is located within a pressure vessel, the pressure vessel is operable to withstand both elevated and reduced pressures (i.e., vacuum). Optionally, before the SAB reactants (314) is introduced to the SAB FBR (310), the pressure within the SAB FBR (310) and within the SAB vessel is less than atmospheric pressure (i.e., the reactor and pressure vessel are under vacuum).

In some examples, the SAB reactants (314) has a ratio of carbon dioxide to hydrogen from about 1:6 to about 1:2, such as about 1:4.

Optionally, the process further includes flowing the gas mixture (which may include both the SAB gas mixture and the SMR gas mixture) through one or more additional FBRs before venting the SMR reactants (114) into the SAB external reactor environment. The additional FBR(s) may have the same specifications as the SAB FBR (310), or may have different specifications than the SAB FBR (310). In various specific examples, any of the three reactions can be carried out first followed by the other two reactions, and the entire series (three reactions) is carried out at least once, and preferably is carried out more than one time. After the first reaction, in each reaction, at least some of the product from the previous reaction is used as a reactant.

When one or more containment vessels are provided, the containment vessel(s) allow the three reactions in the series to be carried out at two or more different times. For example, as described elsewhere herein, endothermic SMR and WGS reactions can be carried out when a source of renewable energy is readily available, and the SAB reactants (314) produced by the WGS reaction can be held in a containment vessel until a later time when energy is needed, but no sufficient source of renewable energy is available to provide that energy. At that later time, the SAB reactants (314) can be accessed and reacted in the exothermic SAB reaction, and the heat from that reaction can be used directly or converted to electricity, thereby providing energy from a renewable energy source. After the SAB reaction, the SMR reactants (114) produced by the SAB reaction can be held in a containment vessel until sufficient renewable energy is available to power the SMR reaction and optionally the WGS reaction to reproduce the SAB reactants (314). If necessary or desired, the WGS reactants (214) produced by the SMR reaction can be stored in a containment vessel for later reaction in the WGS reaction and conversion to SAB reactants (314).

The following examples of temperatures and pressures used in the reactions are given as guidelines only, and persons skilled in the art will understand how to adjust conditions based on their particular requirements.

In some cases, the SAB reaction includes increasing the temperature of the internal volume of the SAB FBR (310) from an initial temperature to a reaction temperature prior to introducing the SAB reactants (314) into the SAB FBR (310). In some examples, the initial temperature is about room temperature (approximately 25° C.). In some examples, the reaction temperature is from about 480° C. to about 550° C. In other examples, the reaction temperature is from about 490° C. to about 540° C., from about 490° C. to about 530° C., from about 500° C. to about 520° C., or from about 500° C. to about 510° C.

In some examples, the SAB reactants (314) has a pressure of at least 60 pounds per square inch (psi) when it is introduced to the SAB FBR (310). In other examples, the SAB reactants (314) has a pressure from about 70 psi to about 4000 psi when it is introduced to the SAB FBR (310). In still other examples, the SAB reactants (314) has a pressure from about 60 psi to about 4000 psi, from about 80 psi to about 4000 psi, from about 100 psi to about 4000 psi, or from about 1000 psi to about 4000 psi when it is introduced to the SAB FBR (310). In some examples, the SAB FBR (310) has a pressure in the interior volume from about 70 psi to about 4000 psi. In still other examples, the SAB FBR (310) has a pressure in the interior volume from about 60 psi to about 4000 psi, from about 80 psi to about 4000 psi, from about 100 psi to about 4000 psi, or from about 1000 psi to about 4000 psi.

In some examples, the SAB reactants (314) has a SAB reactants (314) initial temperature from about 18° C. to about 550° C. when the SAB reactants (314) is introduced to the SAB FBR (310). In other examples, the SAB reactants (314) initial temperature is from about 18° C. to about 30° C. or is from about 20° C. to about 25° C. when the SAB reactants (314) is introduced to the SAB FBR (310). In some examples, the SAB reactants (314) is heated prior to being introduced into the SAB FBR (310). Thus, in other examples still, the SAB reactants (314) initial temperature is from about 490° C. to about 520° C. or is from about 500° C. to about 510° C. when the SAB reactants (314) is introduced to the SAB FBR (310).

In some examples, flowing the SAB reactants (314) through the SAB FBR (310) includes flowing the SAB reactants (314) at a rate of at least 1,000 cubic feet per minute (cfm). In other examples, flowing the SAB reactants (314) through the SAB FBR (310) includes flowing the SAB reactants (314) at a rate of at least 1200, at least 1300, at least 1400, at least 1500, at least 1600, at least 1700, at least 1800, at least 1900, or at least 2000 cfm. In some examples, the SAB reactants (314) flows through the SAB FBR (310) at a rate of 10,000 to 100,000 cfm. In other examples, the SAB reactants (314) flows through the SAB FBR (310) at a rate of 20,000 to 80,000 cfm; 40,000 to 60,000 cfm; 10,000 to 50,000 cfm, or 50,000 to 100,000 cfm. In still other examples, the SAB reactants (314) flows through the SAB FBR (310) at a rate of 100,000 to 1,000,000 cfm, 200,000 to 800,000 cfm; 400,000 to 600,000 cfm; 100,000 to 500,000 cfm, or 500,000 to 1,000,000 cfm.

In some examples, the pressure in the external reactor environment will increase as the SMR reactants (114) flows into the external reactor environment. In some examples, the initial pressure of the external reactor environment is from about 0 to about 14 psi. In some examples, the final pressure of the external reactor environment is at least 100 psi. In other examples, the final pressure of the external reactor environment is from about 100 psi to about 4000 psi or from about 500 psi to about 4000 psi.

In some examples, the chemical reaction in the SAB FBR (310) generates heat that increases or maintains the temperature of the internal volume of the SAB FBR (310). In some examples, after the step of increasing the temperature of the internal volume of the SAB FBR (310) to the SAB reaction temperature, the process does not include controlling the temperature of the internal volume of the SAB FBR (310). In some examples, the process does not include using a heat exchanger. In some examples, the heat generated by the reaction maintains the temperature of the internal volume of the SAB FBR (310) between about 500° C. and about 520° C. or between about 480° C. and about 550° C.

In other examples, the process further includes controlling the temperature of the internal volume of the SAB FBR (310) using a heat exchanger. In some examples, the heat exchanger maintains the temperature of the internal volume of the SAB FBR (310) at or below about 550° C. The temperature of the SAB FBR (310) is the SAB reaction temperature. In some examples, the SAB reaction temperature is at least 400° C., at least 450° C., at least 500° C., or at least 510° C. In some examples, the SAB reaction temperature is from about 400° C. to about 520° C. In some examples, the SAB reaction temperature is no more than about 520° C. In other examples, the SAB reaction temperature is no more than about 510° C., about 505° C., or about 500° C.

In some examples, the process further includes removing the SMR reactants (114) from the external reactor environment. In some examples, the SMR reactants (114) has a temperature from about 400° C. to about 550° C. when it is removed from the external reactor environment. In some examples, the SMR reactants (114) has a pressure from about 60 psi to about 4000 psi when it is removed from the external reactor environment. In some examples, the SMR reactants (114) is removed from the external reactor environment at a flow rate that maintains the pressure of the external reactor environment from about 60 to about 4000 psi.

In some examples, the process further comprises introducing the SMR reactants (114) to the internal volume of the SMR FBR (110) through a conduit extending from a point external to the second pressure vessel to a point interior to the SMR FBR (110), so the SMR reactants (114) is isolated from the second external reactor environment. In some examples, the SMR reactants (114) flows through the SMR FBR (110) at a rate of 10,000 to 100,000 cfm. In other examples, the SMR reactants (114) flows through the SMR FBR (110) at a rate of 20,000 to 80,000 cfm; 40,000 to 60,000 cfm; 10,000 to 50,000 cfm, or 50,000 to 100,000 cfm. In still other examples, the SMR reactants (114) flows through the SMR FBR (110) at a rate of 100,000 to 1,000,000 cfm, 200,000 to 800,000 cfm; 400,000 to 600,000 cfm; 100,000 to 500,000 cfm, or 500,000 to 1,000,000 cfm. In some examples the flow rate through the SMR FBR (110) is approximately equal to the flow rate through the SAB FBR (310). In other examples, the flow rate through the SMR FBR (110) is not equal to the flow rate through the SAB FBR (310).

In some examples, the SMR reactants (114) comprises methane and water vapor. In some examples, the SMR reactants (114) has a pressure of at least 70 pounds per square inch (psi) when it is introduced to the SMR FBR (110). In other examples, the SMR reactants (114) has a pressure from about 70 psi to about 4000 psi when it is introduced to the SMR FBR (110). In still other examples, the SMR reactants (114) has a pressure from about 60 psi to about 4000 psi, from about 80 psi to about 4000 psi, from about 100 psi to about 4000 psi, or from about 1000 psi to about 4000 psi when it is introduced to the SMR FBR (110). In some examples, the SMR FBR (110) has a pressure in the interior volume (i.e., second external reactor environment) from about 70 psi to about 4000 psi. In still other examples, the SMR FBR (110) has a pressure in the interior volume from about 60 psi to about 4000 psi, from about 80 psi to about 4000 psi, from about 100 psi to about 4000 psi, or from about 1000 psi to about 4000 psi.

In some examples, the process further includes heating the second external reactor environment defined by the interior of the second pressure vessel, such as by adding a hot gas, so that the SMR FBR (110) is heated by the gas, but the interior volume of the SMR FBR (110) is not in direct contact with the hot gas. In some examples, the hot gas has a temperature of at least about 800° C. when it is introduced to the second external reactor environment inside the second pressure vessel. In some examples, the hot gas has a temperature from about 800° C. to about 880° C. when it is introduced to the second external reactor environment. In other examples, the hot gas has a temperature from about 810° C. to about 870° C., from about 820° C. to about 870° C., or from about 830° C. to about 860° C. when it is introduced to the second external reactor environment.

In some examples, the process further comprises increasing the temperature of the second external reactor environment from an initial temperature to a final temperature by introduction of the hot gas. The hot gas may flow into the interior of the second pressure vessel (i.e., second external reactor environment) at an inlet and flow out of the interior of the second pressure vessel at an outlet. In some examples, the hot gas maintains the temperature of second external reactor environment at about 800° C. to about 880° C. In other examples, the temperature of second external reactor environment is from about 810° C. to about 870° C., from about 820° C. to about 860° C., from about 800° C. to about 840° C., or from about 840° C. to about 880° C. As the hot gas flows out of the interior of the second pressure vessel at an outlet, the hot gas stream (or portions thereof) may be routed to one or more of the accumulator(s), the steam generator, and the third pressure vessel.

In some examples, the WGS reactants (214) is at a temperature of about 800° C. to about 880° C. when it is introduced to the WGS FBR (210). In some examples, the WGS FBR (210) is at a temperature of about 400° C. to about 1000° C. when the WGS reactants (214) is introduced to the WGS FBR (210). When the WGS reactants (214) is introduced to the WGS FBR (210), the interior of the third pressure vessel (i.e., third external reactor environment) is at a pressure of about 290 to 500 PSI. The third pressure vessel is operable to withstand both elevated and reduced pressures (i.e., vacuum).

In some examples, the WGS reactants (214) flows through the WGS FBR (210) at a rate of 10,000 to 100,000 cfm. In other examples, the WGS reactants (214) flows through the WGS FBR (210) at a rate of 20,000 to 80,000 cfm; 40,000 to 60,000 cfm; 10,000 to 50,000 cfm, or 50,000 to 100,000 cfm. In still other examples, the WGS reactants (214) flows through the WGS FBR (210) at a rate of 100,000 to 1,000,000 cfm, 200,000 to 800,000 cfm; 400,000 to 600,000 cfm; 100,000 to 500,000 cfm, or 500,000 to 1,000,000 cfm. In some examples the flow rate through the WGS FBR (210) is approximately equal to the flow rate through the SMR FBR (110). In other examples, the flow rate through the WGS FBR (210) is not equal to the flow rate through the SMR FBR (110).

In some examples, steam is introduced into the third external reactor environment. In some cases, the steam is generated from burning hydrogen and water in a separate vessel. The steam flows into the third external reactor environment at an inlet, and out of the third external reactor environment at an outlet. In some examples, the steam is at a temperature of about 400° C. to about 1000° C. when the steam is introduced to the third external reactor environment at the inlet. In other examples, plasma generated from water is used as a source of steam. In some examples, the heat generated by exothermic reaction in the WGS FBR (210) heats the steam in the third external reactor environment. When the steam leaves the third external reactor environment, it may flow to the accumulator, or through a steam turbine, or a portion of the steam may flow to the accumulator while another portion may flow to the steam turbine. In addition, a portion may flow to the WGS FBR (210), along with the WGS reactants (214).

The process further includes using heat produced by the exothermic reactions (e.g., from one or more of the SMR reactants (114), the WGS reactants (214), or the SAB reactants (314), or heat from the hot gas after it heats the SMR reaction to produce or increase the temperature of steam.

### Examples

In the following examples, unless otherwise specified, gases are at 0° C. (32° F.) and 100 kPa (1 bar), referred to as standard temperature and pressure (“STP”), and one mole of gas has a volume of 22.4 L.

### Example 1: Reactions Used in the Method

Reaction I: CH₄+2H₂O→CO+3H₂+H₂O

In Reaction I, 22.4 L (16 g) of methane is combined with 44.8 L (36 g) of water (as steam) to yield 22.4 L (28 g) of carbon monoxide, 67.2 L (6 g) of hydrogen, and 22.4 L (18 g) of water (as steam). The reaction is carried out at a temperature of 700-1000° C. and a pressure of 14-40 bar. The standard enthalpy of reaction (ΔH°_{r×n}) is +206 kJ/mole, so carrying out this reaction as specified requires thermal energy in an amount of 206 kJ, which is equivalent to 0.05722 kWh of electrical energy

The energy required for this endothermic reaction can be supplied as thermal energy or as electrical energy, and can be supplied from a renewable energy source, such as but not limited to wind, solar, hydropower, etc. The result of this reaction is stored potential energy in the form of the product gases that can be reacted in a subsequent exothermic reaction to produce thermal energy and/or electrical energy via turbine.

Reaction II: CO+3H₂+H₂O→CO₂+4H₂

In Reaction II, 22.4 L (28 g) of carbon monoxide is combined with 67.2 L (6 g) of hydrogen and 18 L (18 g) water (as steam) to yield 22.4 L (44 g) of carbon dioxide and 89.6 L (8 g) of hydrogen. The reaction is carried out at a temperature of 310-450°. The standard enthalpy of reaction (ΔH°_{r×n}) is −41 kJ/mole, so carrying out this reaction as specified releases thermal energy in an amount of 41 kJ, which is equivalent to 0.01139 kWh of electrical energy

Reaction III: CO₂+4H₂→CH₄+2H₂O

In Reaction III, 22.4 L (44 g) of carbon dioxide is combined with 89.6 L (8 g) of hydrogen to yield 44.8 L (36 g) of water (as steam) and 22.4 L (16 g) methane. The reaction takes place at 500° C. The standard enthalpy of reaction (ΔH°_{r×n}) is −165 kJ/mole, so carrying out this reaction as specified produces thermal energy in an amount of 165 kJ, which is equivalent to 0.045833 kWh of electrical energy.

### Example 2

Reaction III (CO₂+4H₂→CH₄+2H₂O) was carried out in a 2 L FBR with a catalyst. After preheating the reactor to 500° C. to activate the catalyst and then totally removing the external heat, carbon dioxide and hydrogen were introduced to the FBR a single line in at a premixed molar ratio of 1:4 at a flowrate of 25 L/min. The reaction was carried out at about 500° C. and about 67 psi. Because of the steady flow and pressure there was no need for temperature control as the reaction was extremely stable, remaining at about 500° C. The reaction produced a product gas mixture that included methane and steam. The product gas mixture exited the FBR at about 500° C. through a pipe that was insulated, except for an insulated portion of the pipe that was routed through an insulated oil bath. As the hot product gas mixture flowed through the uninsulated portion of the pipe, the oil bath came to a steady state temperature of about 75-78° C. in about an hour. The product gas mixture exiting the oil bath through the pipe had a reduced temperature due to transferring heat to the oil. The chemically formed water was condensed via a knockout drum and isolated from the remainder of the product gas.

The methane flowed through a further conduit to a Bunsen burner, where it was burned, producing an open and consistent flame throughout the experiment. The consistent flame from the Bunsen burner throughout the experiment signified a consistent flow, which was further corroborated by the inline flowmeter

The example demonstrates the ability of the methods to provide a heat from a source that is at a substantially constant temperature of up to 500° C. without carbon dioxide emissions. Such heat can be used to drive electricity production, distillation, and/or other types of processes and reactions requiring heat.

### Example 3: Potential Applications of the Technology

A gas mixture including CO₂ and H₂ at a ratio of 1:4 is transported through a pipeline to a use site. Alternatively, in other similar examples, the gas mixture can be transported to the use site by pipeline, rail, tube trailer, or any other suitable transportation method, or combination of methods. At the use site, the gas mixture is introduced to a system comprising Reactions I, II, and III, where the reactions, in combination, convert the gas mixture to heat, on a cyclic basis if desired, potable water, and/or useful hydrocarbon gases, fuels and chemicals. This schema allows for the efficient transport of “stored” renewable energy to other locations. This would be true if the gas was piped from a high solar or wind area where the hydrogen in particular, was synthesized from electrolysis and then piped to an area where these renewable resources are not readily available. Also, since the reaction yields potable water, this would be a way to provide extra water particularly to an area suffering from drought or under stress from forest fires or other natural disasters.

Another application is to use the heat in a radiator type of arrangement in a HVAC/Hot Water System. In this application, ambient air would be passed through series of pipes containing the 500 C.° steam and methane mixture evolving from a FBR as previously discussed in reaction (III). This heat would kill viruses (including corona viruses like Covid-19), bacteria, and funguses as well. The remaining heat could be used to heat or cool (via absorption chillers) a building. Also, via heat exchangers, hot water could be supplied as well. A system such as this would be useful in a hospital or nursing home setting to help in reducing the pathogen load in the air, while at the same time reducing the cost of hot water and heating/air conditioning requirements. The natural gas could be saved and recycled off site for further use, and any remaining water could be used to irrigate landscaping around a hospital or nursing home in this example.

Another application utilizing the heat produced would be to distill/desalinate water. Also, the heat could be used to distill water/alcohol mixtures or other appropriate chemical mixtures. It is important to understand the flexibility of the system and how it can be optimized for a desired output.

In other applications, the closed loop of reactions (I, II and III) as previously discussed, could be opened after reaction (II) providing access to CO₂ and 4H₂. In this instance, the hydrogen and CO₂ could be distilled such that the CO₂ is liquified and the hydrogen remains a gas. The liquid CO₂ could be used to cool large banks of servers in a data center or “miners′ in a bitcoin farm. In the case of Bitcoin mines, cooling the “miners” reduces electrical costs by 20% and extends the life of the electronics. The CO₂ could be recycled back to liquid again, and the hydrogen could be used for a variety of purposes.

Another application of this technology would be to open the closed loop after reaction (I). In this resulting reaction, CO and 3H₂ are obtained. Using these reactants, it then becomes possible to perform Fischer-Tropsch synthesis. This of course can lead to the production of many fuels such as gasoline, diesel, and jet fuel. Also, other chemicals such as ethylene could be synthesized as well. Also precursors for drugs could be a possibility.

Although several applications have been shown, this technology has applications anywhere a reliable heat source is needed without CO₂ emissions, to clean fuels. If CO₂ is recycled and hydrogen is obtained from renewable energy, this technology would go a long way to reducing the carbon footprint while still benefitting from the value that carbon chemistry not fossil fuels bring to our society.

The following paragraphs describe examples of methods and systems described herein.

Paragraph 1. A method for generating and storing energy for use on demand, the method comprising (i) carrying out a series of reactions consisting of Reactions I, II, and III

CH₄+H₂O→CO+3H₂ (I)

CO+H₂O→CO₂+H₂ (II)

CO₂+4H₂→CH₄+2H₂O (III)

wherein any of Reaction I, II, or III can be a first reaction in the series, provided when Reaction I is the first reaction in the series, at least some of the CO produced in Reaction I is reacted in Reaction II, and at least some of the CO₂ produced in Reaction II is reacted in Reaction III; when Reaction II is the first reaction in the series, at least some of the CO₂ produced in Reaction II is reacted in Reaction III, and at least some of the CH₄ produced in Reaction III is reacted in Reaction I; and when Reaction III is the first reaction in the series, at least some of the CH₄ produced in Reaction III is reacted in Reaction I, and at least some of the CO produced in Reaction I is reacted in Reaction II; and repeating the first reaction in the series of reactions.

Paragraph 2. The method of any preceding paragraph, wherein Reaction I is the first reaction in the series of reactions, and wherein step ii comprises reacting at least some of the CH₄ produced in Reaction III in Reaction I.

Paragraph 3. The method of any preceding paragraph, wherein Reaction II is the first reaction in the series of reactions, and wherein step ii comprises reacting at least some of the CO produced in Reaction I in Reaction II.

Paragraph 4. The method of any preceding paragraph, wherein Reaction III is the first reaction in the series of reactions, and wherein step ii comprises reacting at least some of the CO₂ produced in Reaction II in Reaction III.

Paragraph 5. The method of any preceding paragraph, further comprising after repeating the first reaction, repeating the second reaction.

Paragraph 6. The method of any preceding paragraph, further comprising after repeating the second reaction, repeating the third reaction.

Paragraph 7. The method of any preceding paragraph, further comprising after repeating the third reaction, repeating the series of reactions one or more times.

Paragraph 8. The method of any preceding paragraph, wherein the method is carried out as a closed loop system.

Paragraph 9. The method of any preceding paragraph, wherein the series of reactions is carried out at least three times as a closed loop system.

Paragraph 10. The method of any preceding paragraph, further comprising storing for a period of time at least a portion of the CO, H₂, CO₂, H₂O, or CH₄ after it is produced by Reaction I, II, or III and before it is reacted in the next reaction, wherein the period of time is at least 6 hours, at least 12 hours, at least 24 hours, or at least 48 hours.

Paragraph 11. The method of any preceding paragraph, wherein at least a portion of the CO or the H₂ is stored after it is produced by Reaction I and before it is reacted in Reaction II.

Paragraph 12. The method of any preceding paragraph, wherein at least a portion of the CO₂ is stored after it is produced by Reaction II and before it is reacted in Reaction III.

Paragraph 13. The method of any preceding paragraph, wherein at least a portion of the CH₄ is stored after it is produced by Reaction III and before it is reacted in Reaction I.

Paragraph 14. The method of any preceding paragraph, further comprising transporting at least a portion of the CO, H₂, CO₂, H₂O, or CH₄ after it is produced by Reaction I, II, or III and before it is reacted in the next reaction, wherein the transporting is over a distance of less than 5 miles, less than 1 mile, less than 500 feet, less than 100 feet.

Paragraph 15. The method of any preceding paragraph, further comprising transporting at least a portion of the CO, H₂, CO₂, H₂O, or CH₄ after it is produced by Reaction I, II, or III and before it is reacted in the next reaction, wherein the transporting is over a distance of more than 5 miles, more than 10 miles, more than 100 miles, or more than 500 miles.

Paragraph 16. The method of any preceding paragraph, wherein at least a portion of the CO or the H₂ is transported after it is produced by Reaction I and before it is reacted in Reaction II.

Paragraph 17. The method of any preceding paragraph, wherein at least a portion of the CO₂ is transported after it is produced by Reaction II and before it is reacted in Reaction III.

Paragraph 18. The method of any preceding paragraph, wherein at least a portion of the CH₄ is transported after it is produced by Reaction III and before it is reacted in Reaction I.

Paragraph 19. The method of any preceding paragraph, further comprising using heat produced by Reaction III in another system, method, or device.

Paragraph 20. The method of any preceding paragraph, further comprising providing heat to initiate or maintain one or more of reactions, wherein the heat is derived from a renewable energy source.

Paragraph 21. A system for generating and storing energy for use on demand, the method comprising (i) three fluidized bed reactors for carrying out a series of three chemical reactions; (ii) three vacuum jacketed vessels, wherein each fluidized bed reactor is inside one of the vacuum jacketed vessels; (iii) at least one plasma generator for providing thermal energy to initiate and/or drive the three chemical reactions; and (iv) at least one turbine generator for receiving thermal energy from at least one hot product gas, wherein the hot product gas is produced by one of the three chemical reactions, and wherein the turbine generator produces electrical energy; wherein the system is configured so the series of three chemical reactions can be carried out at least one time as a closed process.

Paragraph 22. The system of any preceding paragraph, further comprising at least one knockout drum for condensing water produced by at least one of the three chemical reactions.

Paragraph 23. The system of any preceding paragraph, comprising at least two, at least three, or at least four turbine generators.

Paragraph 24. The system of any preceding paragraph, further comprising at least one electrolyzer for producing hydrogen, wherein the electrolyzer is powered by renewable energy.

Paragraph 25. The system of any preceding paragraph, further comprising at least one heat exchanger for receiving thermal energy from at least one hot product gas, wherein the hot product gas is produced by one of the three chemical reactions.

The subject matter of embodiments of the present invention is described herein with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

1. A method for generating and storing energy for use on demand, the method comprising
(i) carrying out a series of reactions consisting essentially of Reactions I, II, and III
CH₄+H₂O→CO+3H₂ (I)
CO+H₂O→CO₂+H₂ (II)
CO₂+4H₂→CH₄+2H₂O (III)
wherein any of Reaction I, II, or III can be a first reaction in the series, provided
when Reaction I is the first reaction in the series, at least some of the CO produced in Reaction I is reacted in Reaction II, and at least some of the CO₂ produced in Reaction II is reacted in Reaction III;
when Reaction II is the first reaction in the series, at least some of the CO₂ produced in Reaction II is reacted in Reaction III, and at least some of the CH₄ produced in Reaction III is reacted in Reaction I; and
when Reaction III is the first reaction in the series, at least some of the CH₄ produced in Reaction III is reacted in Reaction I, and at least some of the CO produced in Reaction I is reacted in Reaction II; and
(ii) repeating the first reaction in the series of reactions.

2. The method of claim 1,
wherein Reaction I is the first reaction in the series of reactions, and wherein step ii comprises reacting at least some of the CH₄ produced in Reaction III in Reaction I; or
wherein Reaction II is the first reaction in the series of reactions, and wherein step ii comprises reacting at least some of the CO produced in Reaction I in Reaction II; or
wherein Reaction III is the first reaction in the series of reactions, and wherein step ii comprises reacting at least some of the CO₂ produced in Reaction II in Reaction III.

3. (canceled)

4. (canceled)

5. The method of claim 1, further comprising after repeating the first reaction, repeating the second reaction.

6. The method of claim 5, further comprising after repeating the second reaction, repeating the third reaction.

7. The method of claim 6, further comprising after repeating the third reaction, repeating the series of reactions one or more times.

8. The method of claim 1, wherein the method is carried out as a closed loop system.

9. The method of claim 8, wherein the series of reactions is carried out at least three times as a closed loop system.

10. The method of claim 1, further comprising storing for at least 48 hours at least a portion of the CO, Hz, CO₂, H₂O, or CH₄ after it is produced by Reaction I, II, or III and before it is reacted in the next reaction.

11. (canceled)

12. (canceled)

13. (canceled)

14. The method of claim 1, further comprising transporting at least a portion of the CO, Hz, CO₂, H₂O, or CH₄ after it is produced by Reaction I, II, or III and before it is reacted in the next reaction, wherein the transporting is over a distance of less than 5 miles, less than 1 mile, less than 500 feet, less than 100 feet.

15. The method of claim 1, further comprising transporting at least a portion of the CO, Hz, CO₂, H₂O, or CH₄ after it is produced by Reaction I, II, or III and before it is reacted in the next reaction, wherein the transporting is over a distance of more than 5 miles, more than 10 miles, more than 100 miles, or more than 500 miles.

16. The method of claim 15, wherein at least a portion of the CO or the H₂ is transported after it is produced by Reaction I and before it is reacted in Reaction II.

17. The method of claim 15, wherein at least a portion of the CO₂ is transported after it is produced by Reaction II and before it is reacted in Reaction III.

18. The method of claim 15, wherein at least a portion of the CH₄ is transported after it is produced by Reaction III and before it is reacted in Reaction I.

19. The method of claim 1, further comprising using heat produced by Reaction III in another system, method, or device.

20. The method of claim 19, further comprising providing heat to initiate or maintain one or more of the reactions, wherein the heat is derived from a renewable energy source.

21. A system for generating and storing energy for use on demand, the method comprising
(i) three fluidized bed reactors for carrying out a series of three chemical reactions consisting essentially of Reactions I, II, and III
CH₄+H₂O→CO+3H₂ (I)
CO+H₂O→CO₂+H₂ (II)
CO₂+4H₂→CH₄+2H₂O (III)
(ii) three vacuum jacketed vessels, wherein each fluidized bed reactor is inside one of the vacuum jacketed vessels;
(iii) at least one plasma generator for providing thermal energy to initiate and/or drive the three chemical reactions; and
(iv) at least one turbine generator for receiving thermal energy from at least one hot product gas, wherein the hot product gas is produced by one of the three chemical reactions, and wherein the turbine generator produces electrical energy;
wherein the system is configured so the series of three chemical reactions can be carried out at least one time as a closed process.

22. The system of claim 21, further comprising at least one knockout drum for condensing water produced by at least one of the three chemical reactions.

23. The system of claim 21, comprising at least two turbine generators.

24. The system of claim 21, further comprising at least one electrolyzer for producing hydrogen, wherein the electrolyzer is powered by renewable energy.

25. The system of claim 24, further comprising at least one heat exchanger for receiving thermal energy from at least one hot product gas, wherein the hot product gas is produced by one of the three chemical reactions.
